(19) United States
(12) Patent Application Publication
(10) Pub. No.: US 2017/0106141 A1
(43) Pub. Date: Apr. 20, 2017

(54) METHOD FOR DETECTION AND DISPLAY OF EXTRAVASATION AND INFILTRATION OF FLUIDS AND SUBSTANCES IN SUBDERMAL OR INTRADERMAL TISSUE

(71) Applicant: THE UNITED STATES OF AMERICA AS REPRESENTED BY THE SECRETARY OF THE AIR FORCE, WRIGHT-PATTERSON, OH (US)

(72) Inventors: ROBERT L. CRANE, KETTERING, OH (US); DAVID M. CALLARD, PALO ALTO, CA (US)

(21) Appl. No.: 15/338,629

(22) Filed: Oct. 31, 2016

Related U.S. Application Data
(63) Continuation of application No. 10/421,270, filed on Apr. 23, 2003, now abandoned.

(51) Int. Cl. A61M 5/168; A61B 5/02; A61B 5/00

(52) U.S. Cl. CPC A61M 5/16836; A61B 5/0059; A61B 5/02042; A61B 5/4839; A61M 2205/3306

(57) ABSTRACT

A method for the real-time visualization and detection of extravasated and or infiltrated fluid and substances, including blood, that occur near the cannulation site of an injection is described wherein illumination or transillumination with near infrared light is used to image the contrast in real-time between absorbing and nonabsorbing subdermal and intradermal structures of blood vessels and remaining surrounding tissue, foreign substances and other structures in order to establish a baseline image of the body area of interest, and any new image is monitored and compared with the baseline image to detect the extravasation and/or infiltration of fluids and substances, including blood, around a vein or artery into the subdermal or intradermal tissue.

### RIGHTS OF THE GOVERNMENT

The invention described herein may be manufactured and used by or for the Government of the United States for all governmental purposes without the payment of any royalty.

### BACKGROUND OF THE INVENTION

The invention relates generally to medical devices and procedures, and more particularly to procedures for the real-time detection and visualization of infiltrated or extravasated substances, including blood, that occur in subdermal or intradermal tissues near the cannulation site of an injection procedure, such as in the intravascular delivery or extraction of various substances or media.

During intravascular administration of substances, a portion of the substances may escape from the interior of the vein or artery into surrounding tissues. Leaking of intravascular fluids or medicine is referred to as infiltration if the substance is limited to causing mild effects such as swelling and may include bleeding. Extravasation describes leaking of intravascular fluids or medicines that may, in worse cases, cause tissue damage; bleeding may also be described as extravasation as it indicates some rupture or failure of the vessels wall(s). Incidents that cause extravasation and infiltration include improper venipuncture, such as a transfixation of a vein, rupture of the vasculature, perhaps due to weakened vascular walls in patients of advanced age, disease states, abrasion by the cannula, or the administration of a toxic agent. Problems with extravasation and infiltration may include bruising, discoloration of the skin or discomfort to the patient, or more serious problems associated with thrombosis, bleeding leading to hypovolemia, and tissue toxicity, such as in the administration of toxic substances associated with chemotherapy wherein the concentration of a toxic substance is carefully monitored to ensure dilution of the substance to appropriate levels during administration. Extravasation into the spaces surrounding a blood vessel poses very serious problems of local tissue toxicity and/or possible necrosis depending upon the agent, and a lack of accurate delivery of metered dosage into the patient's vascular system.

Non-invasive detection of extravasation and/or infiltration in the prior art is accomplished chiefly by medical personnel visualizing a swelling in the region with an unaided eye or manually palpating the swelling or by assessment of patient's complaint of discomfort, pressure, swelling, or pain. In such cases, large amounts of extravasated fluids and/or blood components may accumulate in the tissue surrounding the vasculature before detection of the condition is made.

Other automatic methods exist for detection of extravasation and/or infiltration using various electronic signal and detection systems. One such method found in U.S. Pat. No. 4,877,034, a photo-plethysmographic technique, discloses a method for detecting a plurality of wavelengths of electromagnetic radiation emitted and then detected as reflected or reemitted radiation at the injection site. Such measurements are taken prior to or in the absence of injection in order to establish a baseline. During injection infiltrated or extravasated fluids cause a shift in the detected radiation that can be measured against the baseline for evidence of infiltration or extravasation.

Another method, U.S. Pat. No. 4,647,281, discloses subcutaneous temperature sensing via an antenna and a microwave radiometer and measurement of the temperature of fluid introduced at an injection site. In this method an alarm is activated when the temperature differential between injected fluid and surrounding tissue reaches a prefigured threshold. Yet other detection techniques rely upon plethysmographic measurements of volume changes in the tissue surrounding an injection and extravasation and/or infiltration site. Changing volumes of subdermal fluid resulting from infiltration may be measured as changes in tissue impedance, deflections of strain cuffs, or changes in pressure sensors at the site.

These prior methods as a whole exhibit significant limitations in causing direct obstructions at the site of injection and extravasation or infiltration thereby preventing visualization and palpation of the tissue, a critical step by the healthcare provider under all circumstances. As an example, U.S. Pat. No. 4,877,034 requires placement of an electromagnetic radiation emitter and detector patch directly over the area of injection and accordingly prevents observation and palpation of the physical signs of extravasation or infiltration.

One method that avoids obstruction of observation and palpation is U.S. Pat. No. 6,459,931 that provides for placement of a first order energy source and receiver in close proximity to the injection site; the receiver measures changes in bulk electrical properties of the tissue and fluid at the infiltration site. This method, along with the other prior art for detection of extravasation, only discloses a technique for generation of electronic signals for processing and interpretation of extravasation or infiltration vents; they do not teach a method of immediate visual display of the actual physical features in the form of contrast images of the extravasating or infiltrating fluids. An unobstructed visual display of extravasation or infiltration offers the primary advantages of instant confirmation and assessment by the healthcare provider including indications of volume, rate, and distribution of extravasating fluids in complement to palpation of the site.

Prior art methods to assist medical personnel in the visualization of veins, arteries and other subcutaneous structures of the body include application of tourniquets, use of a flashlight, direct application of liquid crystalline materials, ultrasound and use of dual fiber optic sources. These methods may indicate vein or artery location, but do not allow for the detection of extravasation or infiltration in direct visual respect to the vasculature. A procedure is therefore needed to detect reliably and non-invasively and to display extravasation and infiltration in real-time, especially at the time a patient is undergoing a procedure, in order to identify and to diagnose the extravasation or infiltration of blood components or other fluids and substances, such as injectable agents, at the time of administration.

Transillumination and reflection imaging in the near infrared (NIR) are non-invasive techniques for detecting the vasculature wherein the passage of light through the body or reflection from its surface and near subsurface regions are used to observe subsurface structures. Using such techniques, a body surface area of interest is illuminated and characteristics, such as light intensity and wavelength reflected or scattered from that area form an image. U.S. Pat. No. 6,230,046 to Crane et al. ('046 patent) teaches a device that illuminates veins and arteries and displays blood vessel structure in-vivo in a non-invasive and painless manner to facilitate insertion or extraction of fluids for medical treatment. However, the device and method taught in the '046 patent does not sense and display the presence of extravasated or infiltrated fluids and substances into subdermal or intradermal tissues. The entire teachings of the '046 patent are incorporated by reference herein.

The invention overcomes limitations of related prior art techniques by providing an easily transportable, non-invasive, real-time method for detecting and displaying extravasation or infiltration of subdermal and/or intradermal tissue without direct obstruction at the site of injection and suspected infiltration or extravasation. In accordance with a principal teaching of the invention, differences in image content are significant and permit detection and visualization of extravasation or infiltration of contrast media, blood, medicines, or other fluids and substances, and the degree of contrast in the NIR are used to quantify the amount of nonabsorbing or absorbing substance being infused and subsequently leaking from the intended vessel.

In a preferred embodiment of the invention, illumination or transillumination with NIR is used to image the contrast in real-time between absorbing and nonabsorbing subdermal and intradermal structures of blood vessels and remaining surrounding tissue, foreign substances and other structures. After baseline images have been established, any new image is monitored and compared with the original to detect the extravastation or infiltration of fluids or blood around a vein or artery and into the subdermal and/or intradermal tissues. The invention therefore allows detection of location, size, depth, direction of movement or flow, rate of movement or flow, shape, constitution, volume of features or other defining aspects.

The invention finds substantial use in medical procedures such as demonstrating the proper technique for inserting an IV catheter, avoiding severe bruising during IV access for medical treatment, correctly administering various medications and imaging contrast agents, monitoring arterial bleeding after removal of a femoral artery catheter and avoiding thrombosis, and monitoring rapid extravasation of high-pressure, injected contrast dyes following vessel rupture.

It is a principal object of the invention to provide system and method for real-time detection and visualization of extravasation and infiltration of fluids and substances in subdermal or intradermal tissues.

It is another object of the invention to provide a method for detection of extravasation and infiltration during a medical procedure.

These and other objects of the invention will become apparent as a detailed description of representative embodiments proceeds.

### SUMMARY OF THE INVENTION

In accordance with the foregoing principles and objects of the invention, a method for the real-time visualization and detection of extravasated or infiltrated fluids and substances, including blood, that occur near the cannulation site of an injection is described wherein illumination or transillumination with NIR is used to image the contrast in real-time between absorbing and nonabsorbing subdermal and intradermal structures of blood vessels and remaining surrounding tissue, foreign substances and other structures in order to establish a baseline image of the body area of interest, and any new image is monitored and compared with the baseline image to detect the extravasation or infiltration of fluid and substances, including blood, around a vein or artery and into the subdermal or intradermal tissue.

### DESCRIPTION OF THE DRAWINGS

The invention will be more clearly understood from the following detailed description of representative embodiments thereof read in conjunction with the accompanying drawings wherein:

FIG. 1 shows schematically a light source, detector, a forearm of a patient and an IV instrument illustrating an embodiment of the invention;

FIG. 2a shows schematically a baseline image of a forearm of a patient as in FIG. 1;

FIG. 2b shows schematically the baseline image of FIG. 2a after a substance transparent to NIR is inserted into a vein of the patient; as in FIG. 1;

FIG. 2c shows schematically the baseline image of FIG. 2a after a substance absorbent of the NIR is inserted into a vein of the patient; and

FIG. 3 shows schematically the forearm of a patient of FIG. 1 after the IV instrument is removed illustrating the presence of extravasation or infiltration around a vein.

### DETAILED DESCRIPTION

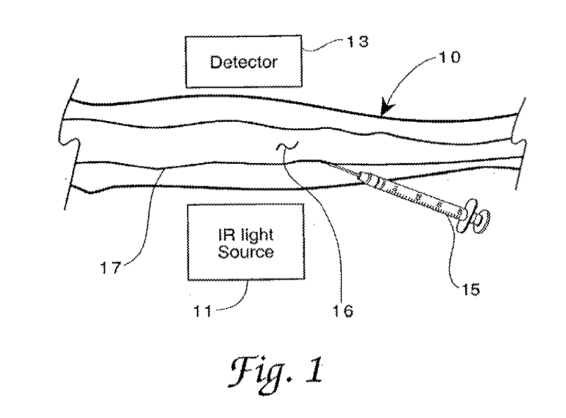

Referring now to the drawings, FIG. 1 illustrates the method of the invention in one of its embodiments for imaging extravasation or infiltration, wherein a surface area of the body, such as forearm 10, is placed near an NIR light source 11 and between source 11 and detector 13 (in the transillumination mode of the invention), in order to perform a particular procedure, such as in the administration of a specific substance into the vasculature (artery or vein 17) using IV instrument 15. Source 11 preferably emits light in the wavelength range of about 0.3 to 1.0 micron, and detector 13 is sensitive to light in that range. Alternatively, source 11 could be placed to directly illuminate forearm 10 such that a reflected NIR image of the infusion area 16 at instrument 15 is viewed. Source 11 can also be placed near the area of interest of forearm 10 such that scattered light is used to illuminate the area of interest. Detector 13 is focused upon the area of interest and does not interfere with the procedure or obstruct medical personnel performing the procedure. NIR energy from source 11 and detected by detector 13 may be that reflected, refracted, absorbed, transmitted or scattered by subdermal and intradermal structures in the body area of interest, such as forearm 10.

Detector 13 may include any of the instruments well known in the art and used for acquiring and displaying NIR images of the body area of interest, such as those described in the '046 patent, including image intensifier tubes (night vision goggles), photomultiplier tubes, photodiodes, silicon based arrays such as charged couple devices (CCD), complementary metal-oxide semiconductor (CMOS), or other solid state devices with appropriate filtering to enhance the signal-to-noise ratio of the image. Detector 13 converts the NIR image to a visible one so that medical personnel may view the infusion procedure in order to properly insert instrument 15 and to detect any extravasation or infiltration of the infusion fluid or substance, including blood, that occurs in the area of interest. The detected energy is selectively filtered to create one or more images of subcutaneous structures in the area. In a preferred embodiment, the detected energy is selectively filtered using a filter having a narrow passband centered substantially on at least one wavelength in the range of about 0.30 to 1.0 micron and more particularly at wavelengths of about 0.32, 0.345, 0.41, 0.43, 0.455, 0.54, 0.56, 0.58, 0.7, 0.76 micron. The image generated from the filtered light is used to determine inherent, baseline, visual characteristics of the subdermal and intradermal tissues in the body area of interest.

In accordance with a principal feature of the invention, a baseline image is first established prior to the initiation of the procedure to be monitored. In a non-limiting example, the administration of a radiographic contrast agent may be monitored. After the baseline image is obtained, detector 13 continues to collect NIR energy from the body area of interest and generates additional images of the area of interest during the procedure. Continued monitoring of the NIR images of the subdermal and intradermal tissues permits the determination of the state of the infusion process. These images are compared to the baseline image to determine changes near the vasculature in the body area of interest that evidence extravasation or infiltration.

An extravasated or infiltrated fluid or substance near a vein or artery is detected by observing the differences in the apparent image densities of the baseline vascular structure and infused substances in the area of interest. The image of the extravasated or infused fluid or substance may be darker or lighter depending on the relative absorbance of the NIR light as compared to the surrounding tissue and vasculature. Darker images are formed where the infusion fluid or substance absorbs or attenuates the NIR light more than the normal tissues as established in the baseline image.

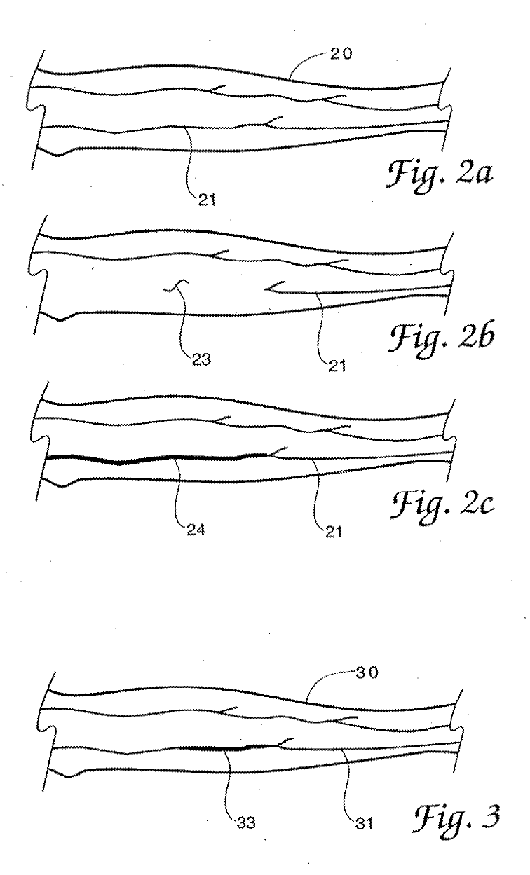

To illustrate the foregoing, reference is now made to FIGS. 2a-c, wherein FIG. 2a shows a drawing of a baseline image of a forearm 20 of a patient as in FIG. 1, FIG. 2b shows the baseline image after a substance transparent to NIR is inserted into vein 21 of the patient, and FIG. 2c shows the baseline image of FIG. 2a after a substance absorbent of the NIR is inserted into vein 21. The extravasation or infiltration of a contrast agent is determined either by the appearance of a lighter region 23 (FIG. 2b) or darker region 24 (FIG. 2c), depending on the transmissivity or absorbance of the infused substance, as compared to the baseline images created for the surrounding subdermal tissues prior to infusion. The representations of the extravasated or infiltrated substances can be any such size and shape depending on a number of factors including volume and rate of substance penetration, patient anatomy and health conditions, size and location of cannulation tool, among others, and therefore the illustrations in FIGS. 2a-b do not limit the invention in terms of specifying the various sizes and shapes of detected substances.

FIG. 3 shows schematically the forearm 30 of a patient after the IV instrument (15 of FIG. 1) is removed from vein 31, illustrating the presence of infiltrated blood 33 around vein 31. Infiltration of blood components is determined by a darkened region image because blood attenuates the NIR light more than the surrounding tissue. Again, the illustration of FIG. 3 does not limit the invention in terms of specifying the various sizes and shapes of detected blood.

The method taught by the invention can therefore be easily used to detect the occurrence of an infiltration and/or extravasation in real-time in a particular body area of interest. According to the invention, the generated images of the subdermal or intradermal tissues may be displayed to the practitioner on a video monitor, heads-up display or other presentation modality. Extravasation and/ or infiltration in the body area of interest may be detected, optically imaged and distinguished in real-time during a procedure without obstruction or interruption of the procedure, and during minor movement of a patient without requiring re-imaging.

The invention therefore provides system and method for real-time visualization and detection of extravasated and/or infiltrated fluids and substances, including blood, that occur near the cannulation site of an injection. It is understood that modifications to the invention may be made as might occur to one with skill in the field of the invention within the scope of the appended claims. All embodiments contemplated hereunder that achieve the objects of the invention have therefore not been shown in complete detail. Other embodiments may be developed without departing from the spirit of the invention or from the scope of the appended claims.

We claim:

1. A method for real-time visualization and detection of extravasted and/or infiltrated fluids and substances, including blood, that occur near the cannulation site of an injection comprising the steps of:
(a) providing a light source in the wavelength range of about 0.3 to about 1.0 micron;
(b) illuminating a portion of the body near the intended site of an injection in at least one of a reflection mode and a transillumination mode;
(c) providing a detector sensitive to light in said wavelength range of said light source for receiving said light transmitted from said portion of the body;
(d) selectively filtering the light received by said detector using a filter having a narrow passband centered substantially on at least one wavelength in said wavelength range of said light source;
(e) generating a first image from the filtered light to provide a baseline image of the subdermal and intradermal tissues in said portion of the body;
(f) generating at least one additional image of said portion of the body during a procedure that includes an injection at said body portion; and
(g) comparing said at least one additional image with said baseline image to detect changes near the vasculature in said portion of the body to detect extravasation or infiltration in said portion of the body.

2. The method of claim 1 wherein said detector includes an image intensifier tube, photomultiplier tube, a photo diode, a charge coupled device, or a CMOS.

3. The method of claim 1 wherein said step of filtering said detected energy is performed using a filter having a narrow passband centered substantially on at least one wavelength selected from the group consisting of 0.32, 0.345, 0.41, 0.43, 0.455, 0.54, 0.56, 0,58, 0.7, 0.76 micron.

4. A method for monitoring a medical procedure that includes an injection procedure at a portion of the body, comprising the steps of:
(a) providing a light source in the wavelength range of about 0.3 to about 1.0 micron;
(b) illuminating a portion of the body near the intended site of an injection in at least reflection mode and a transillumination mode;
(c) providing a detector sensitive to light in said wavelength range of said light source for receiving said light transmitted from said portion of the body;
(d) selectively filtering the light received by said detector using a filter having a narrow passband centered substantially on at least one wavelength in said wavelength range of said light source;
(e) generating a series of images from the filtered light of the subdermal and intradermal tissues in said portion of the body during the medical procedure; and
(f) comparing said images to observe changes near the vasculature in said portion of the body and to detect extravasation and/or infiltration in said portion of the body.

5. The method of claim 4 wherein said detector includes an image intensifier tube, photomultiplier tube, a photo diode, a charge coupled device, or a CMOS.

6. The method of claim 4 wherein said step of filtering said detected energy is performed using a filter having a narrow passband centered substantially on at least one wavelength selected from the group consisting of 0.32, 0.345, 0.41, 0.43, 0.455, 0.54, 0.56, 0.58, 0.7, 0.76 micron.
